(19) United States
(12) Patent Application Publication
(10) Pub. No.: US 2024/0123265 A1
(43) Pub. Date: Apr. 18, 2024

(54) MASK CONTROLLING AIR VOLUME AND CONTROLLING METHOD THEREFOR

(71) Applicant: Samsung Electronics Co., Ltd., Suwon-si (KR)

(72) Inventors: Hyunah KIM, Suwon-si (KR); Seonuk NA, Suwon-si (KR); Joonhyoung KIM, Suwon-si (KR); Jeeyong KIM, Suwon-si (KR); Hyojin KIM, Suwon-si (KR); Youngchal PARK, Suwon-si (KR); Hyeongjoon SEO, Suwon-si (KR); Youngtae SONG, Suwon-si (KR); Kisup LEE, Suwon-si (KR); Sanghoon LEE, Suwon-si (KR); Sungjune CHO, Suwon-si (KR); Sungjin CHO, Suwon-si (KR); Younglae JO, Suwon-si (KR); Wangbyung CHAE, Suwon-si (KR)

(21) Appl. No.: 18/518,189

(22) Filed: Nov. 22, 2023

Related U.S. Application Data
(63) Continuation of application No. PCT/KR2023/015652, filed on Oct. 11, 2023.

(30) Foreign Application Priority Data
Oct. 12, 2022 (KR) 10-2022-0130867

(51) Int. Cl. A62B 18/00; A62B 9/00; A62B 18/10; A62B 23/02

(52) U.S. Cl. CPC A62B 18/003; A62B 9/006; A62B 18/10; A62B 23/02

(57) ABSTRACT

A mask and a controlling method thereof are disclosed. The mask includes a fan that provides an air volume to an inside of the mask, a valve that discharges air from the mask, a pressure sensor, and a processor. The processor may control the pressure sensor to detect a maximum pressure value and a minimum pressure value inside the mask worn by a user. The processor may identify a time for a single breath based on a maximum pressure value and a minimum pressure value detected at the pressure sensor. The processor may identify a number of breaths based on the identified time for the single breath and a predetermined time. The processor may control the fan to provide an air volume set at a level corresponding to the identified number of breaths among a plurality of levels divided based on a predetermined number of breaths.

### CROSS-REFERENCE TO RELATED APPLICATIONS

This application is a bypass continuation of International Application No. PCT/KR2023/015652, filed on Oct. 11, 2023, which is based on and claims priority to Korean Patent Application No. 10-2022-0130867, filed on Oct. 12, 2022, in the Korean Intellectual Property Office, the disclosures of which are incorporated by reference herein in their entireties.

### BACKGROUND

### 1. Field

The disclosure relates to a mask controlling an air volume.

### 2. Description of Related Art

A mask may be used for protecting a respiratory organ from foreign substances such as dust, and preventing an infectious disease that is infected through a respiratory organ. A mask may be made of a fiber material. Also, a filter may be added to block fine particles. In general, manual type masks consisting of cotton and a filter are being widely used. A user wearing a manual type mask can breathe by overcoming the pressure of the filter only with his or her breathing.

Recently, active type masks providing an air volume to the insides of the masks are being developed. An active type mask may include a fan and a valve. An active type mask may provide external air to the inside of the mask through a fan, and discharge the air inside the mask through a valve.

### SUMMARY

A mask according to one or more embodiments of the disclosure may include a fan configured to provide an air volume to the inside of the mask, a valve configured to discharge air from inside the mask to the outside, a sensor including a pressure sensor, and at least one processor. The at least one processor may control the pressure sensor to detect pressure including the maximum pressure value and the minimum pressure value inside the mask worn by a user. The at least one processor may identify a time for a single breath based on a point of the maximum pressure value and a point of the minimum pressure value detected at the pressure sensor. The at least one processor may identify the number of breaths based on the identified time for the single breath and first a predetermined time. The at least one processor may control the fan to provide an air volume set at a level corresponding to the identified number of breaths among a plurality of levels divided based on a predetermined number of breaths to the inside of the mask.

A controlling method for a mask according to one or more embodiments of the disclosure may include the step of detecting pressure including the maximum pressure value and the minimum pressure value inside the mask worn by a user. The controlling method for a mask may include the step of identifying one time of breathing based on the detected point of the maximum pressure value and the detected point of the minimum pressure value. The controlling method for a mask may include the step of identifying the number of breaths based on the identified one time of breathing and a first predetermined time. The controlling method for a mask may include the step of providing an air volume set at a level corresponding to the identified number of breaths among a plurality of levels that are divided based on a predetermined number of breaths to the inside of the mask.

A controlling method for a mask according to one or more embodiments of the disclosure may include the step of acquiring an internal temperature and an external temperature of the mask. The controlling method for a mask may include the step of controlling an air volume provided to an inside of the mask based on a temperature difference between the acquired internal temperature and the acquired external temperature.

### BRIEF DESCRIPTION OF THE DRAWINGS

FIG. 1 is a diagram illustrating a mask according to one or more embodiments of the disclosure;

FIG. 2 is a diagram illustrating an air flow channel inside a mask according to one or more embodiments of the disclosure;

FIG. 3 is a block diagram illustrating a configuration of a mask according to one or more embodiments of the disclosure;

FIG. 4 is a block diagram illustrating a detailed configuration of a mask according to one or more embodiments of the disclosure;

FIG. 5 is a diagram illustrating the pressure inside a mask according to one or more embodiments of the disclosure;

FIG. 6 is a diagram illustrating an embodiment of controlling an air volume provided to the inside of a mask;

FIG. 7 is a flow chart illustrating a process of controlling an air volume according to one or more embodiments of the disclosure;

FIG. 8A to FIG. 8C are diagrams illustrating an embodiment of changing a boundary value of an air volume level;

FIG. 9 and FIG. 10 are diagrams illustrating an embodiment of adjusting an air volume based on a temperature difference; and

FIG. 11 is a flow chart illustrating a controlling method for a mask according to one or more embodiments of the disclosure.

### DETAILED DESCRIPTION

Hereinafter, various embodiments will be described in more detail with reference to the accompanying drawings. The embodiments described in this specification may be modified in various ways. Also, specific embodiments may be illustrated in the drawings, and described in detail in the detailed description. However, specific embodiments disclosed in the accompanying drawings are just for making the various embodiments easily understood. Accordingly, the technical idea of the disclosure is not restricted by the specific embodiments disclosed in the accompanying drawings, and the embodiments should be understood as including all equivalents or alternatives included in the idea and the technical scope of the disclosure.

Also, terms including ordinal numbers such as ‘the first’ and ‘the second’ may be used to describe various components, but these components are not limited by the aforementioned terms. The aforementioned terms are used only for the purpose of distinguishing one component from another component.

In addition, in this specification, terms such as “include” and “have” should be construed as designating that there are such characteristics, numbers, steps, operations, elements, components, or a combination thereof described in the specification, but not as excluding in advance the existence or possibility of adding one or more of other characteristics, numbers, steps, operations, elements, components, or a combination thereof. Further, the description in the disclosure that an element is “coupled with/to” or “connected to” another element should be interpreted to mean that the one element may be directly coupled with/to or connected to the another element, but still another element may exist between the elements. In contrast, the description that one element is “directly coupled” or “directly connected” to another element can be interpreted to mean that still another element does not exist between the one element and the another element.

Meanwhile, “a module” or “a part” for the elements used in this specification performs at least one function or operation. Also, “a module” or “a part” may perform a function or an operation by hardware, software, or a combination of hardware and software. Also, a plurality of “modules” or a plurality of “parts” except “a module” or “a part” that needs to be implemented in specific hardware or is performed in at least one processor may be integrated into at least one module. Further, singular expressions include plural expressions, unless defined obviously differently in the context.

Also, in the description of the disclosure, the order of each step should be understood in a nonrestrictive way, unless a preceding step should necessarily be performed prior to a subsequent step in a logical and temporal sense. That is, excluding an exceptional case as above, even if a process described as a subsequent step is performed prior to a process described as a preceding step, there would be no influence on the essence of the disclosure, and the scope of the disclosure should also be defined regardless of the orders of steps. Further, the description “A or B” in this specification is defined to include not only a case wherein one of A or B is selectively referred to, but also a case wherein both of A and B are included. In addition, the term “include” in this specification includes a case wherein elements other than elements listed as being included are further included.

Further, in this specification, only essential elements necessary for describing the disclosure are described, and elements not related to the essence of the disclosure are not mentioned. Also, the descriptions of the disclosure should not be interpreted to have an exclusive meaning of including only the elements mentioned, but to have a non-exclusive meaning of also including other elements.

In addition, in describing the disclosure, in case it is determined that detailed explanation of related known functions or features may unnecessarily confuse the gist of the disclosure, the detailed explanation will be abridged or omitted. Meanwhile, each embodiment of the disclosure may be independently implemented or operated, but it may also be implemented or operated in combination with another embodiment.

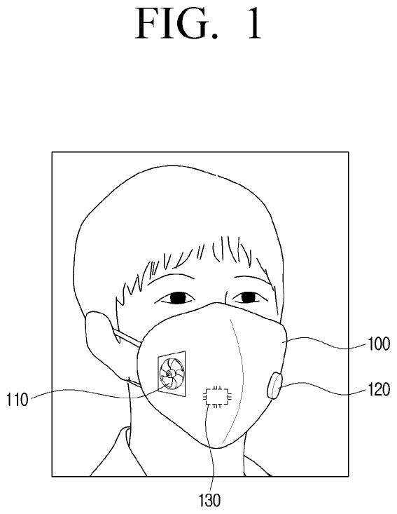

FIG. 1 is a diagram illustrating a mask according to one or more embodiments of the disclosure.

Referring to FIG. 1, a mask 100 is illustrated. The mask 100 may include a fan 110, a valve 120, and a processor 130. The mask 100 according to the disclosure may mean an active type mask (a mechanical type mask) that provides the external air to the inside of the mask through the fan 110, and discharges the air inside the mask through the valve 120.

The fan 110 may provide an air volume to the inside of the mask 100. In the mask 100, a part that is implemented as a fiber material, and covers the respiratory organ (e.g., a nose, a mouth) of the user may be referred to as the main body. As an example, the fan 110 may be located on one side surface of the main body of the mask 100. The fan 110 may rotate by the number of rotations (e.g., rotations per minute (RPM)) set by the processor 130. According to the rotation of the fan 110, the air outside the mask may be provided to the inside of the mask 100.

The valve 120 may discharge the air inside the mask 100 to the outside of the mask 100. As an example, the valve 120 may be located on the other side surface of the main body of the mask 100. However, the aforementioned locations of the fan 110 and the valve 120 are merely an example, and they may be arranged in various locations. The valve 120 may be a manual type valve. In this case, when the user inhales, the valve 120 may be closed, and when the user exhales, the valve 120 may be opened and discharge the air inside the mask 100 to the outside of the mask 100. Alternatively, the valve 120 may be an active type valve. In this case, the valve 120 may identify breathing of the user, and may be opened or closed based on the identified breathing of the user.

The processor 130 may control the driving and the number of rotations of the fan 110. If the valve 120 is an active type valve, the processor 130 may control the opening and closing of the valve 120. The mask 100 may include one or more processors 130. The processor 130 may be implemented on one board together with other electronic components and a circuit. Alternatively, the processor 130 may be implemented as one chip together with other electronic components and a circuit. The board or chip including the processor 130 may be located on the main body of the mask 100. The main body of the mask 100 may be implemented as a form wherein a plurality of surfaces are laminated, and the board may be located on the outermost surface of the main body wherein the plurality of surfaces are laminated or between the plurality of surfaces.

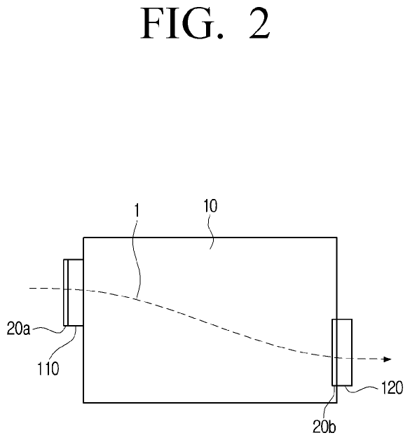

FIG. 2 is a diagram illustrating an air flow channel inside a mask according to one or more embodiments of the disclosure.

Referring to FIG. 2, an air flow channel 1 in the inside 10 of the mask 100 is illustrated. As described above, the mask 100 may include a fan 110 and a valve 120. Also, the mask 100 may include filters 20a, 20b in each of the areas of the fan 110 and the valve 120.

When a user wears the mask 100, the boundary surface of the mask 100 and the user's face may be adjoined. Accordingly, most air flow channels in the worn mask 100 may be formed of routes continued to the fan 110, the inside 10 of the mask, and the valve 120.

The filters 20a, 20b may be arranged on an air flow channel 1 continued to the fan 110, the inside 10 of the mask, and the valve 120. For example, the filters 20a, 20b may include a first filter 20a and a second filter 20b. The first filter 20a may be arranged upstream from the location of the fan 110. That is, the first filter 20a may be arranged in the front end part of the fan 110 suctioning the external air on the air flow channel 1. The first filter 20a may filter fine particles in the air. The second filter 20b may be located downstream from the location of the fan 110, and upstream from the location of the valve 120. That is, the second filter 20b may be arranged in the front end part of the valve 120 discharging the air on the air flow channel 1. The second filter 20b may filter droplets so that the droplets in the inside 10 of the mask is not discharged to the outside.

So far, the configuration of the mask 100 according to one or more embodiments was described. Hereinafter, the electronic configuration of the mask 100 will be described.

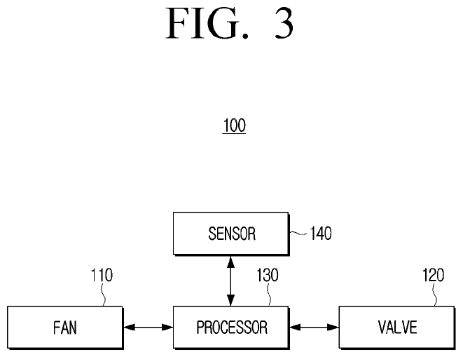

FIG. 3 is a block diagram illustrating a configuration of a mask according to one or more embodiments of the disclosure.

Referring to FIG. 3, the mask 100 may include a fan 110, a valve 120, a processor 130, and a sensor 140. The fan 110 and the valve 120 may be the same as what was described in FIG. 1.

The processor 130 may control each component of the mask 100. For example, the processor 130 may control the fan 110 to adjust an air volume provided to the inside of the mask, and control the sensor 140 to detect user information or the ambient information to acquire information for controlling the fan 110. Alternatively, if the valve 120 is an active type valve, the processor 130 may control the opening and closing of the valve 120.

The processor 130 may control the sensor 140 to detect pressure including the maximum pressure value and the minimum pressure value inside the mask 100. The processor 130 may identify the cycle of breathing based on the detected maximum pressure value and minimum pressure value. Then, the processor 130 may identify the number of breaths based on the identified breathing cycle and a predetermined time. The processor 130 may control the fan 110 to provide the air volume of an operation level corresponding to the identified number of breaths among operation levels of the fan 110 that are divided into a plurality of levels to the inside of the mask 100. Alternatively, the processor 130 may identify the number of breaths based on the number of times of opening and closing of the valve in a specific situation, and control the operation level of the fan 110. Alternatively, the processor 130 may turn off the power of the mask 100 or the fan 110 based on the identified number of breaths and the predetermined time. Alternatively, the processor 130 may set the state of the mask 100 or the fan 110 as an idle state.

The processor 130 may control the sensor 140 to detect the temperature inside the mask 100. Alternatively, the processor 130 may control the sensor 140 to detect the temperature outside of the mask 100. The processor 130 may adjust the set air volume based on the temperature difference between the temperatures inside and outside the mask 100. Alternatively, the processor 130 may change the set number of breaths that divides the operation levels of the fan 110 according to a user's instruction.

The sensor 140 may detect information related to the user or information on the ambient environment. For example, the sensor 140 may include a pressure sensor, and detect the pressure inside the mask 100. The processor 130 may identify the number of breaths of the user based on the detected pressure inside the mask 100. Then, the processor 130 may control the fan 110 to provide the air volume set based on the identified number of breaths of the user to the inside of the mask 100. Alternatively, the sensor 140 may detect the number of opening and closing of the valve 120. Depending on cases, the processor 130 may identify the number of breaths of the user based on the number of opening and closing of the valve. Also, the sensor 140 may include a temperature sensor, and detect the temperature inside the mask 100. Alternatively, the sensor 140 may also detect the temperature outside the mask 100 together. The processor 130 may adjust the set air volume based on the difference between the temperature outside the mask and the temperature inside the mask 100.

For example, the sensor 140 may include a pressure sensor, a temperature sensor, an image sensor, a tracking sensor, an angle sensor, an acceleration sensor, a gravity sensor, a gyro sensor, a geomagnetic sensor, a direction sensor, a motion recognition sensor, a proximity sensor, a voltmeter, an ammeter, a barometer, a hygrometer, an illumination sensor, a heat detection sensor, a touch sensor, an infrared sensor, an ultrasonic sensor, etc.

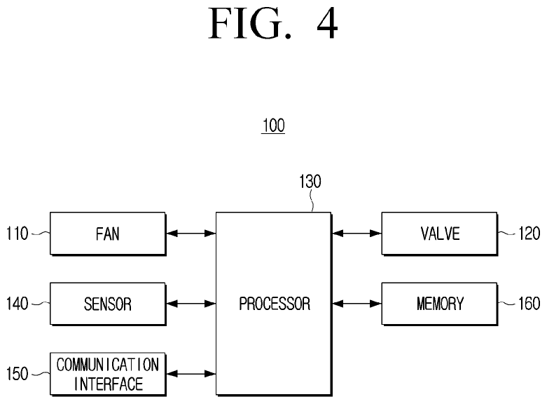

FIG. 4 is a block diagram illustrating a detailed configuration of a mask according to one or more embodiments of the disclosure.

Referring to FIG. 4, the mask 100 may include a fan 110, a valve 120, a processor 130, a sensor 140, a communication interface 150, and a memory 160. The fan 110, the valve 120, and the sensor 140 may be the same as what was illustrated in FIG. 3.

The communication interface 150 may perform communication with an external device. The communication interface 150 may receive a control instruction or external temperature information from the external device. The processor 130 may change the set value of the air volume control level according to the control instruction received from the communication interface 150. Alternatively, the processor 130 may control the air volume provided to the inside of the mask based on the external temperature information received from the communication interface 150 or the information on the detected temperature inside the mask 100.

For example, the communication interface may perform communication with an external device by at least one communication method among communication methods such as Wi-Fi, Wi-Fi Direct, Bluetooth, Zigbee, 3rd Generation (3G), 3rd Generation Partnership Project (3GPP), and Long Term Evolution (LTE). The communication interface may also be referred to as a communication device, a communicator, a communication module, a transceiver, etc.

The memory 160 may store data, an algorithm, etc. for performing the functions of the mask 100, and store programs, instructions, various kinds of information, etc. driven at the mask 100. For example, the algorithm, the data, etc. stored in the memory 160 may be loaded on the processor 130 by control by the processor 130, and perform a data processing process. For example, the memory 160 may be implemented in types such as a ROM, a RAM, an HDD, an SSD, a memory card, etc.

The mask 100 may include all of the aforementioned components, or may include some components. Also, the mask 100 may further include other components performing various functions other than the aforementioned components.

Hereinafter, a process of identifying the number of breaths and controlling the air volume will be explained in detail.

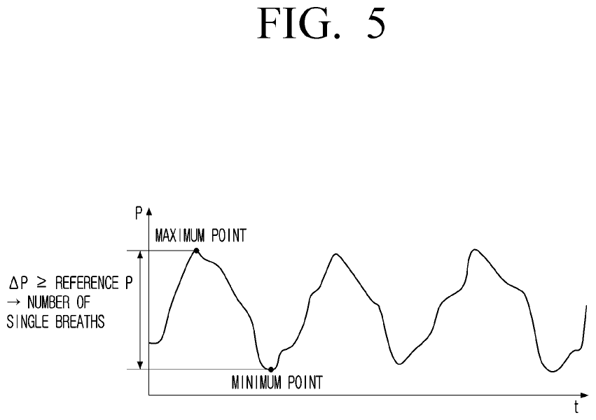

FIG. 5 is a diagram illustrating the pressure inside a mask according to one or more embodiments of the disclosure.

Referring to FIG. 5, a graph of the pressure inside a mask detected at the pressure sensor is illustrated.

The mask 100 may detect the pressure inside the mask 100 by using the pressure sensor. The pressure inside the mask 100 may be detected in real time. When the user inhales, the pressure inside the mask 100 may decrease, and when the user exhales, the pressure inside the mask 100 may increase. The mask 100 may identify the maximum pressure value and the maximum pressure point from the detected pressure inside the mask 100. Also, the mask 100 may identify the minimum pressure value and the minimum pressure point. The mask 100 may identify the number of single breaths based on the point of the maximum pressure and the point of the minimum pressure. Also, the mask 100 may identify a single breath only in a case where the difference between the maximum pressure value and the minimum pressure value is greater than or equal to a threshold value for preventing an error in measurement of the total number of breaths. The mask 100 may identify the number of breaths identified during a predetermined time, and determine the number of breaths as the number of breaths of the user.

For example, if the maximum pressure value is 0.7 kPa, and the minimum pressure value is 0.2 kPa, and the threshold value of a pressure difference is 0.3 kPa, the difference between the maximum pressure value and the minimum pressure value is 0.5 kPa which is greater than the threshold value. Accordingly, the mask 100 may identify the interval between the point of the maximum pressure value and the point of the minimum pressure value as a single breath. Also, if the predetermined time is one minute, the mask 100 may identify the number of breaths during one minute. If the number of breaths of the user during one minute is 15 times, the mask 100 may identify 15 times as the number of breaths of the user.

Alternatively, the mask 100 may identify a single breath by the aforementioned method. Then, the mask 100 may identify the identified a time for the single breath as the cycle of breathing or a breathing cycle, and identify the number of breaths of the user based on the predetermined time and the identified breathing cycle. For example, if the point of the maximum pressure value is one second, and the point of the minimum pressure value is six seconds, the mask 100 may identify the breathing cycle of the user as five seconds. Then, the mask 100 may determine the number of breaths of the user as 12 times based on the predetermined time of one minute and the identified breathing cycle of five seconds.

The mask 100 may determine whether there is an abnormality in the detected pressure. For example, if the detected maximum pressure value or the detected minimum pressure value exceeds a predetermined normal range, the mask 100 may determine that there is an abnormality in the detected pressure. As an example, the mask 100 may identify the normal pressure range as a range between 0.1 kPa and 0.9 kPa. If the detected maximum pressure value exceeds 0.9 kPa or the detected minimum pressure value is smaller than or equal to 0.1 kPa, the mask 100 may determine that there is an abnormality in the detected pressure. When the mask 100 determines an abnormality in the pressure, the mask 100 may identify the number of breaths based on the number of times of opening and closing of the valve 120. For example, the valve 120 may be a manual type valve. If the valve 120 opens, the mask 100 may determine that the user is in a state of exhaling, and if the valve 120 closes, the mask 100 may determine that the user is in a state of inhaling. Accordingly, the mask 100 may determine one time of breathing of the user based on the state of one time of opening/closing of the valve 120. The mask 100 may identify the number of breaths based on the predetermined time and a time from a single breath. Alternatively, the mask 100 may determine the cycle for a time of opening and closing of the valve 120 as the breathing cycle, and identify the number of breaths based on the determined breathing cycle.

The mask 100 may control the air volume provided to the inside of the mask 100 based on the identified number of breaths.

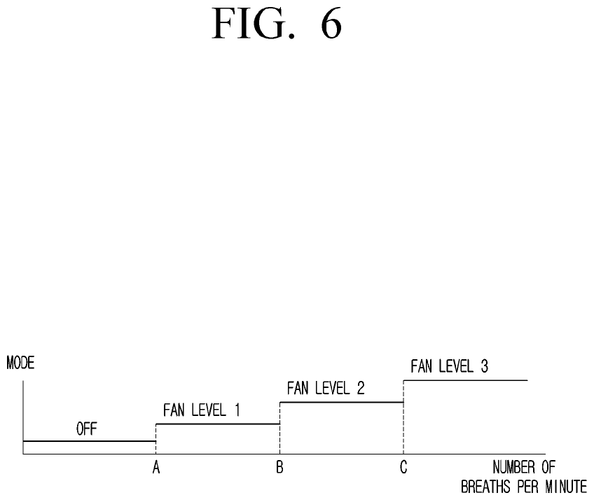

FIG. 6 is a diagram illustrating an embodiment of controlling an air volume provided to the inside of a mask 100.

Referring to FIG. 6, an example of the number of breaths and the control levels of the air volume is illustrated. The mask 100 may control the air volume in a plurality of levels based on the number of breaths. For example, if the number of breaths per minute is smaller than 10 breaths, the mask 100 may turn off the fan 110. For example, turning off the fan 110 may mean setting the state of the fan 110 to a standby state wherein the fan 110 does not rotate (e.g., the number of rotations is 0), and it may also mean turning off the power of the fan 110. Alternatively, the mask 100 may set the mask 100 in a standby state or turn off the power of the mask 100.

If the number of breaths per minute is greater than or equal to 10 times and smaller than 16 times, the mask 100 may rotate the fan 110 by the number of rotations or RPM corresponding to the first level. If the number of breaths per minute is greater than or equal to 16 times and smaller than 30 times, the mask 100 may rotate the fan 110 by the number of rotations corresponding to the second level. If the number of breaths per minute is greater than or equal to 30 times, the mask 100 may rotate the fan 110 by the number of rotations corresponding to the third level. As an example, the mask 100 may set the number of rotations corresponding to the first level as 60 times per minute, the number of rotations corresponding to the second level as 120 times per minute, and the number of rotations corresponding to the third level as 240 times per minute. Meanwhile, the aforementioned number of breaths and number of rotations are merely an example, and they may be set as various values according to the user, the environment, etc.

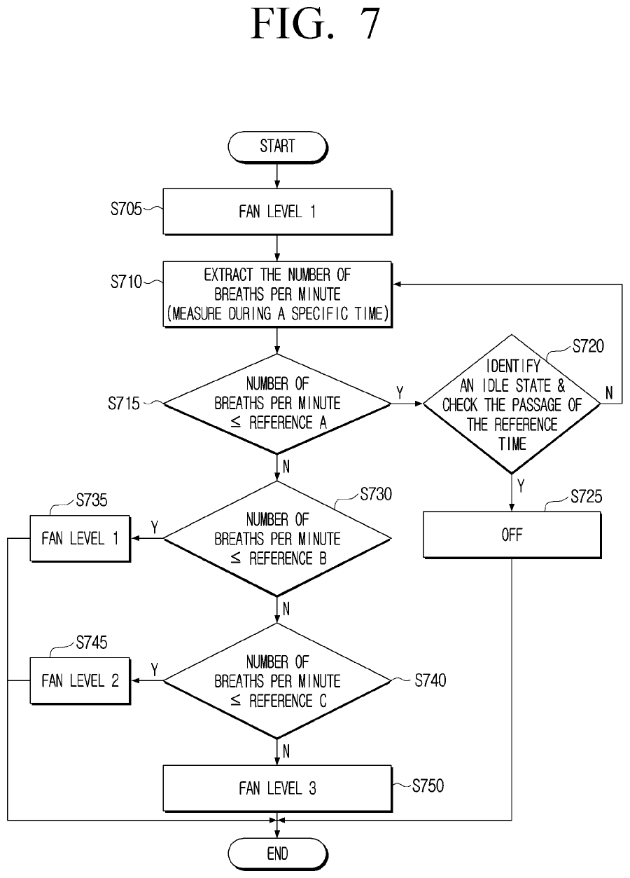

FIG. 7 is a flow chart illustrating a process of controlling an air volume according to one or more embodiments of the disclosure.

When the mask 100 is turned on, the mask 100 may control the fan 110 to be driven in the first level in operation S705. As an example, the number of rotations of the fan 110 corresponding to the first level may be 60 times per minute. Accordingly, when the mask 100 is turned on, the mask 100 may control the fan 110 to rotate by 60 times per minute.

The mask 100 may identify the number of breaths of the user who is wearing the mask in operation S710. For example, the mask 100 may detect the pressure inside the mask for a specific time, and identify the number of breaths (e.g., the number of breaths per minute) based on the detected pressure. Alternatively, the mask 100 may determine the cycle of breathing based on the detected pressure, and identify the number of breaths based on the determined cycle of breathing. If the mask 100 determines that there is an abnormality in the detected pressure based on the predetermined normal range of pressure, the mask 100 may identify the number of breaths based on the number of times of opening and closing of the valve 120. Alternatively, the mask 100 may determine the opening/closing cycle of the valve 120, and identify the number of breaths based on the determined opening/closing cycle.

The mask 100 may control the driving of the fan 110 in a plurality of levels. For example, the mask 100 may be set so as to drive the fan 110 in an OFF level if the number of breaths is smaller than or equal to A times, and in the first level if the number of breaths exceeds A times and is smaller than B times, and in the second level if the number of breaths exceeds B times and is smaller than C times, and in the third level if the number of breaths exceeds C times.

The mask 100 may determine whether the determined number of breaths is smaller than or equal to A times in operation S715. If the identified number of breaths is smaller than or equal to A times, the mask 100 may determine whether the time during which the identified number of breaths is smaller than or equal to A times is maintained during the predetermined time or longer (identification of an idle state) in operation S720. For example, A times may be set as 10 times, and the predetermined time may be five minutes. The mask 100 may determine whether the number of breaths is maintained as smaller than or equal to 10 times during five minutes. If the number of breaths is smaller than A times during the predetermined time or longer, the user may not be wearing the mask 100. Accordingly, the mask 100 may turn off the power in operation S725. For example, the mask 100 may turn off the power of the fan 110 and the mask 100.

If the identified number of breaths exceeds A times, the mask 100 may determine whether the identified number of breaths is smaller than or equal to B times in operation S730. If the identified number of breaths is smaller than or equal to B times, the mask 100 may drive the fan 110 in the first level in operation S735. As an example, B times may be set as 16 times. Accordingly, if the identified number of breaths exceeds 10 times and is smaller than or equal to 16 times, the mask 100 may control the fan 110 to rotate 60 times per minute.

If the identified number of breaths exceeds B times, the mask 100 may determine whether the identified number of breaths is smaller than or equal to C times in operation S740. If the identified number of breaths is smaller than or equal to C times, the mask 100 may drive the fan 110 in the second level in operation S745. As an example, C times may be set as 30 times, and the number of rotations of the fan 110 corresponding to the second level may be set as 120 times per minute. Accordingly, if the identified number of breaths exceeds 16 times and is smaller than or equal to 30 times, the mask 100 may control the fan 110 to rotate 120 times per minute.

If the identified number of breaths exceeds C times, the mask 100 may drive the fan 110 in the third level in operation S750. As an example, the number of rotations of the fan 110 corresponding to the third level may be set as 240 times per minute. Accordingly, if the identified number of breaths exceeds 30 times, the mask 100 may control the fan 110 to rotate 240 times per minute.

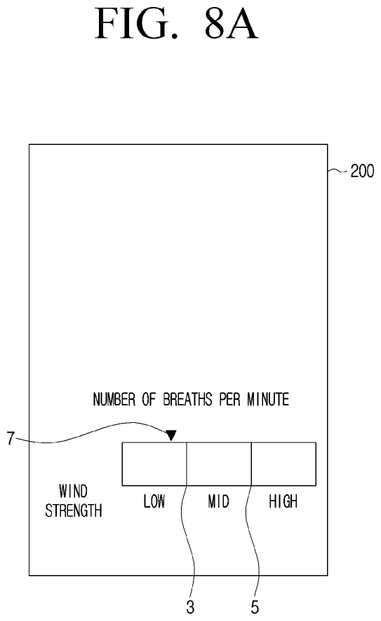

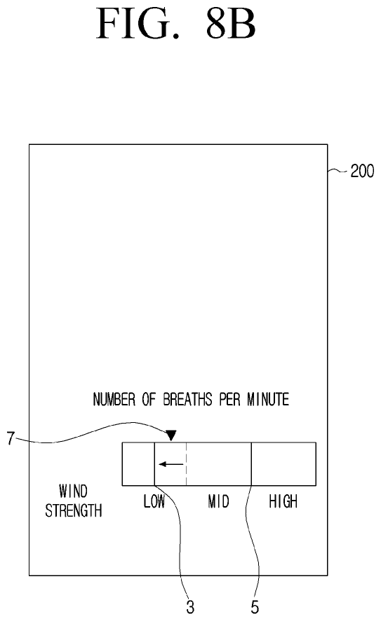

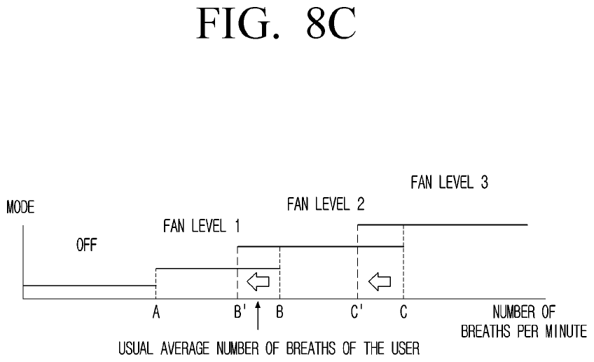

FIG. 8A to FIG. 8C are diagrams illustrating an embodiment of changing a boundary value of an air volume level.

Referring to FIG. 8A, a terminal device 200 wherein information of the mask 100 is displayed is illustrated. For example, the terminal device 200 may include a smartphone, a tablet PC, a laptop computer, a navigation, a slate PC, a wearable device, etc. Also, the terminal device 200 may include an input interface, a communication interface, a camera, a microphone, a speaker, a display, a memory, a sensor, and a processor.

The input interface may receive input of a control instruction from a user. Also, the input interface may receive input of an instruction for changing the boundary value of the air volume level from a user. For example, the input interface receiving input of a user's instruction may be implemented as a keyboard, a button, a keypad, a touch pad, a touch screen, etc. The input interface may also be referred to as an input device, an inputter, an input module, etc.

The communication interface may perform communication with the mask 100. For example, the terminal device 200 may transmit information to the mask 100 or receive information from the mask 100 through the communication interface. The communication interface may receive information on each level of the fan 110, information on the air volume, the number of breaths, etc. from the mask 100. Then, the communication interface may transmit information on the external temperature or information of changing the boundary value of the air volume level to the mask 100. For example, the communication interface may perform communication with an external device by at least one communication method among communication methods such as W-Fi, Wi-Fi Direct, Bluetooth, Zigbee, 3rd Generation (3G), 3rd Generation Partnership Project (3GPP), and Long Term Evolution (LTE). Also, the communication interface may include a GPS module, and acquire location information of a user (or, a terminal device). The communication interface may receive information on the external temperature of the current location from the external device based on the acquired location information. The communication interface may also be referred to as a communication device, a communicator, a communication module, a transceiver, etc.

The camera may photograph the ambient environment of the terminal device 200. Alternatively, the camera may photograph a user's facial expression, operation, gaze, etc. The processor may perform a control operation based on the information of the photographed ambient area or the user's information. For example, the camera may include a CCD sensor and a CMOS sensor. Also, the camera may include an RGB camera and a depth camera.

The microphone may receive input of an ambient sound. Alternatively, the microphone may receive input of a user's voice. The processor may recognize a control instruction based on the input voice of the user, and perform a control operation corresponding to the recognized control instruction.

The speaker outputs a sound signal for which signal processing was performed. For example, the speaker may output a user's input instruction, information related to the state of the mask 100, or information related to an operation, etc. as a voice or a notification sound.

The display may display information by a visual method. For example, as illustrated in FIG. 8A, the display may display an air volume level (e.g., low, mid, high), the boundary line 3, 5 of each level, an indicator 7 indicating the number of breaths, information on the current air volume, etc. The air volume level may be the driving level of the fan 110. As an example, the number of breaths indicating the boundary between low and mid of the air volume may be 16 times, the number of breaths indicating the boundary between mid and high of the air volume may be 30 times, and the number of breaths of the user may be 13 times. Accordingly, the indicator 7 indicating the number of breaths may be located in the low area, and the air volume provided by the mask 100 to the inside may be the low level.

For example, the display may be implemented as a liquid crystal display (LCD), organic light emitting diodes (OLED), a touch screen, etc. In case the display is implemented as a touch screen, the terminal device 200 may receive input of a control instruction through the touch screen.

The memory may store data, an algorithm, etc. performing the functions of the terminal device 200, and store programs, instructions, etc. driven at the terminal device 200. Alternatively, the memory may store information received from the mask 100, information on the external temperature, etc. The algorithm, the data, etc. stored in the memory may be loaded on the processor by control by the processor, and perform a data processing process. For example, the memory may be implemented in types such as a ROM, a RAM, an HDD, an SSD, a memory card, etc.

The sensor may detect information related to the user or the ambient environment. The processor may perform a control operation based on the detected information. Also, the sensor may detect the external temperature of the current location. For example, the sensor may include an image sensor, a tracking sensor, an angle sensor, an acceleration sensor, a gravity sensor, a gyro sensor, a geomagnetic sensor, a direction sensor, a motion recognition sensor, a proximity sensor, a voltmeter, an ammeter, a barometer, a hygrometer, a thermometer, an illumination sensor, a heat detection sensor, a touch sensor, an infrared sensor, an ultrasonic sensor, etc.

The processor may control each component of a terminal device. The processor may be included as one or a plurality of processors. The processor may control the input interface to receive input of a control instruction from the user, and control the communication interface to transmit and receive data (or, information) with the mask 100 and/or an external device. Also, the processor may control the display to display the air volume information and the number of breaths of the mask 100, and control the sensor to detect the external temperature.

The terminal device 200 may include all of the aforementioned components, or may include some components. Also, the terminal device 200 may further include other components performing various functions other than the aforementioned components.

Referring to FIG. 8B, an example of adjusting the boundary value of the air volume level is illustrated. As explained in FIG. 8A, the number of breaths indicating the boundary between low and mid of the air volume may be 16 times, the number of breaths indicating the boundary between mid and high of the air volume may be 30 times, and the number of breaths of the user may be 13 times. However, the user may hope that a bigger air volume is provided to the inside of the mask 100. The user may touch the boundary line of the air volume displayed on the terminal device 200 to move the boundary line. As an example, the user may move the boundary line 3 between low and mid to the low area. As the boundary line 3 between low and mid moves, the number of breaths indicating the boundary between low and mid of the air volume may be changed. For example, the previous number of breaths of 16 times may be changed to 10 times. Even if the number of breaths of the user is identical as 13 times, the air volume may be provided in the mid level according to moving of the boundary line 3. The terminal device 200 may directly receive input of the boundary number of breaths from the user and change the boundary level of the air volume, other than a touch method through the touch screen.

The terminal device 200 may transmit information on the changed boundary value of the air volume to the mask 100. The mask 100 may change the driving level of the fan 110 based on the received information.

Referring to FIG. 8C, an example wherein the driving level of the fan 110 was changed is illustrated. The mask 100 may receive information on the change of the boundary number of breaths from the terminal device 200. Then, the mask 100 may change the boundary value of the driving level of the fan 110 based on the received information on the change. As illustrated in FIG. 8C, the number of breaths of the boundary value between the first level and the second level may have been set as B times, and the number of breaths of the boundary value between the second level and the third level may have been set as C times. The mask 100 may change B times to B′ times, and change C times to C′ times based on the information received from the terminal device 200.

Alternatively, the mask 100 may change the air volume (or, the number of rotations of the fan) based on a temperature difference.

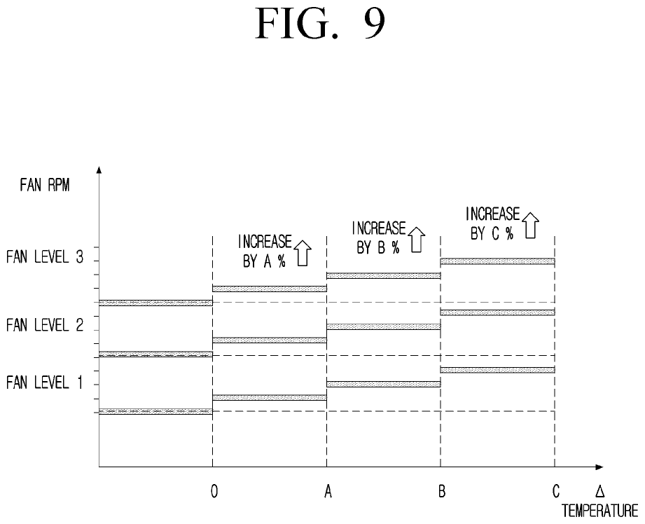

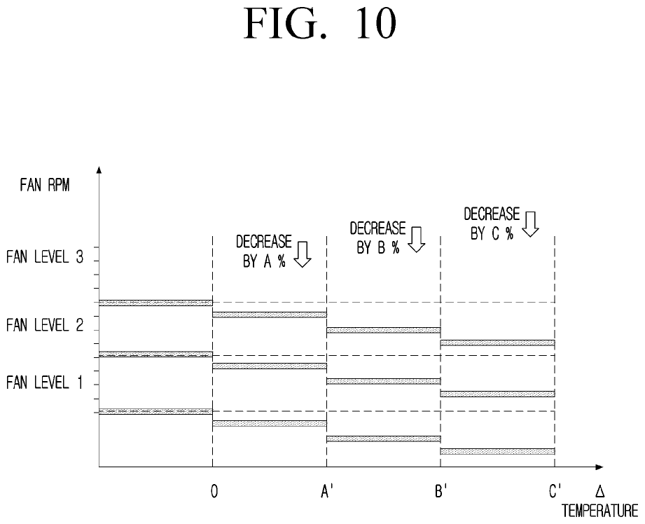

FIG. 9 and FIG. 10 are diagrams illustrating an embodiment of adjusting an air volume based on a temperature difference. Referring to FIG. 9, an example of increasing the air volume is illustrated, and referring to FIG. 10, an example of decreasing the air volume is illustrated.

The mask 100 may detect the temperature inside the mask 100 by using the sensor 140. Then, the mask 100 may acquire the external temperature. For example, the mask 100 may further include a sensor 140 detecting the external temperature and detect the external temperature. Alternatively, the mask 100 may receive information on the external temperature from the terminal device 200. The terminal device 200 may include a first sensor for detecting the external temperature, and detect the external temperature by using the sensor included in the terminal device 200, and transmit information on the detected external temperature to the mask 100. The mask 100 may acquire the information on the external temperature detected at the terminal device 200. Alternatively, the terminal device 200 may acquire the current location information of the user by using a GPS. Then, the terminal device 200 may transmit the acquired current location information to an external device (e.g., a server, a cloud, etc.). The external device may acquire the temperature information based on the received location information, and transmit the information to the terminal device 200. The terminal device 200 may transmit the temperature information received from the external device to the mask 100. The temperature information received by the terminal device 200 may be information on the external temperature of the region wherein the user wearing the mask 100 is located. Accordingly, the mask 100 may acquire the information on the external temperature from the terminal device 200. The mask 100 may acquire a difference between the internal temperature and the external temperature.

The mask 100 may increase or decrease the air volume based on the temperature difference. As illustrated in FIG. 9, the mask 100 may increase the set air volume if the temperature difference is smaller than or equal to a predetermined value. Also, as illustrated in FIG. 10, the mask 100 may decrease the set air volume if the temperature difference exceeds the predetermined value.

For example, the temperature inside the mask 100 may be similar to the body temperature of the user, and may be less influenced by the external temperature. That is, the temperature inside the mask 100 may be a temperature around approximately 36 degrees. The external temperature in summer may be approximately between 28 degrees and 40 degrees. The external temperature in winter may be approximately between −20 degrees and 5 degrees. However, the aforementioned temperatures are merely examples, and the external temperature may be various values according to regions.

In the case of summer, as the weather is hot, the user may feel pleasant when a bigger air volume is provided. In the case of winter, the user may feel unpleasant due to the cold air provided to the inside of the mask 100, and thus the user may feel pleasant when a smaller air volume is provided. Accordingly, the mask 100 of the disclosure may control the number of rotations of the fan 110 so that a bigger air volume is provided in summer, and a smaller air volume is provided in winter.

In the aforementioned example, a difference of the internal temperature based on the external temperature may be approximately between −8 degrees and 4 degrees in summer. Accordingly, if the temperature difference is −10 degrees or higher (Td≥−10), the mask 100 may increase the air volume. Alternatively, the absolute value of the temperature difference may be approximately between 4 degrees and 8 degrees. In this case, the mask 100 may increase the air volume if the temperature difference is 3 degrees or higher and 10 degrees or lower.

As illustrated in FIG. 9, the mask 100 may maintain the control level of the number of rotations of the fan 110 according to the number of breaths, and increase the air volume of each level. Here, the mask 100 may increase the air volume of the first level by a %, increase the air volume of the second level by b %, and increase the air volume of the third level by c %.

Alternatively, in the aforementioned example, a difference of the internal temperature based on the external temperature may be approximately between −56 degrees and −31 degrees in winter. Accordingly, if the temperature difference is −25 degrees or lower (Td≤−25), the mask 100 may decrease the air volume. Alternatively, the absolute value of the temperature difference may be approximately between 31 degrees and 56 degrees. In this case, the mask 100 may decrease the air volume if the temperature difference is 30 degrees or higher.

As illustrated in FIG. 10, the mask 100 may maintain the control level of the number of rotations of the fan 110 according to the number of breaths, and decrease the air volume of each level. Here, the mask 100 may decrease the air volume of the first level by a %, decrease the air volume of the second level by b %, and decrease the air volume of the third level by c %.

So far, various embodiments of the mask 100 controlling an air volume were explained. Hereinafter, a controlling method for the mask 100 will be explained.

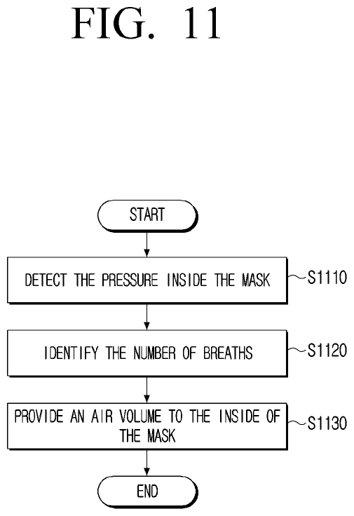

FIG. 11 is a flow chart illustrating a controlling method for a mask according to one or more embodiments of the disclosure.

Referring to FIG. 11, the mask 100 may detect pressure including a maximum pressure value and a minimum pressure value inside the mask 100 worn by a user in operation S1110. Then, the mask 100 may identify a time for a single breath based on the detected point of the maximum pressure value and the detected point of the minimum pressure value. Then, the mask may identify the number of breaths based on the identified time for the single breath and a predetermined time in operation S1120. Alternatively, if the mask 100 identifies that there is an abnormality in the detected pressure, the mask 100 may identify the number of breaths based on the number of opening and closing of the valve. For example, if the detected maximum pressure value or the detected minimum pressure value exceeds a predetermined normal range, the mask 100 may identify that there is an abnormality in the detected pressure.

The mask 100 may provide an air volume set at a level corresponding to the identified number of breaths among a plurality of levels that are divided based on a predetermined number of breaths to the inside of the mask in operation S1130. If the identified number of breaths lasts for a predetermined time or longer at smaller than a predetermined minimum value, the mask may turn off the power of the fan. Alternatively, the mask 100 may turn off the power of the mask 100.

If the mask 100 receives information of changing a predetermined number of breaths dividing the plurality of levels from a terminal device, the mask 100 may change the predetermined number of breaths based on the received information.

Alternatively, the mask 100 may control the air volume based on a difference between the external temperature and the internal temperature. The mask 100 may detect the internal temperature of the mask 100. Then, the mask 100 may acquire the external temperature. For example, the mask 100 may detect the external temperature. Alternatively, the mask 100 may receive the external temperature of the region wherein the user is located from the terminal device. The mask 100 may change the set air volume based on the temperature difference between the acquired external temperature and the detected internal temperature. For example, if the temperature difference is smaller than or equal to a predetermined value, the mask 100 may increase the set air volume, and if the temperature difference is greater than or equal to a predetermined value, the mask may decrease the set air volume.

The mask 100 of the disclosure may provide an air volume based on the number of breaths of the user. Also, the mask 100 may adjust the air volume level according to the user, and adjust the air volume according to the temperature difference so that the user can feel pleasant.

Meanwhile, the effects of the disclosure are not limited to the effects mentioned above, and other effects that were not mentioned could be clearly understood by a person skilled in the art from the following descriptions.

The controlling method for a mask 100 according to the aforementioned various embodiments may be provided as a computer program product. A computer program product may include a software program itself, or a non-transitory computer readable medium having a software program stored therein.

A non-transitory computer readable medium refers to a medium that stores data semi-permanently, and is readable by machines, but not a medium that stores data for a short moment such as a register, a cache, and a memory. Specifically, the aforementioned various applications or programs may be provided while being stored in a non-transitory computer readable medium such as a CD, a DVD, a hard disk, a blue-ray disk, a USB, a memory card, a ROM and the like.

Also, while preferred embodiments of the disclosure have been shown and described, the disclosure is not limited to the aforementioned specific embodiments, and it is apparent that various modifications may be made by those having ordinary skill in the technical field to which the disclosure belongs, without departing from the gist of the disclosure as claimed by the appended claims. Further, it is intended that such modifications are not to be interpreted independently from the technical idea or prospect of the disclosure.

What is claimed is:

1. A mask comprising:
a fan configured to provide an air volume to an inside of the mask;
a valve configured to discharge air from the inside the mask to an outside of the mask;
a sensor including a pressure sensor; and
at least one processor,
wherein the at least one processor is configured to:
control the pressure sensor to detect pressure including a maximum pressure value and a minimum pressure value inside the mask worn by a user,
identify a time for a single breath based on a point of the maximum pressure value and a point of the minimum pressure value detected at the pressure sensor, and identify a number of breaths based on the identified time for the single breath and a first predetermined time, and
control the fan to provide an air volume set at a level corresponding to the identified number of breaths among a plurality of levels that are divided based on a predetermined number of breaths to the inside of the mask.

2. The mask of claim 1, wherein the at least one processor is configured to:
based on identifying an abnormality in the detected pressure, identify the number of breaths based on the number of opening and closing of the valve.

3. The mask of claim 2, wherein the at least one processor is configured to:
based on the detected maximum pressure value or the detected minimum pressure value exceeding a predetermined normal breathing range, identify that there is an abnormality in the detected pressure.

4. The mask of claim 1, wherein the at least one processor is configured to:
based on the identified number of breaths lasting for a second predetermined time or longer below a predetermined minimum value, turn off power to the fan.

5. The mask of claim 1, further comprising:
a communication interface configured to perform communication with a terminal device,
wherein the at least one processor is configured to:
based on receiving information of changing the predetermined number of breaths for dividing the plurality of levels from the terminal device, change the predetermined number of breaths based on the received information.

6. The mask of claim 1, further comprising:
a communication interface configured to perform communication with a terminal device,
wherein the sensor comprises a temperature sensor, and
wherein the at least one processor is configured to:
control the communication interface to receive an external temperature of a region wherein the user is located from the terminal device,
control the temperature sensor to detect an internal temperature of the mask, and
change a set air volume based on a temperature difference between the received external temperature and the detected internal temperature.

7. The mask of claim 1, further comprising:
a communication interface configured to perform communication with a terminal device,
wherein the sensor comprises a temperature sensor, and
wherein the at least one processor is configured to:
control the temperature sensor to detect an external temperature of the mask and an internal temperature of the mask, and
change a set air volume based on a temperature difference between the detected external temperature and the detected internal temperature.

8. The mask of claim 7, wherein the at least one processor is configured to:
based on the temperature difference being smaller than or equal to a predetermined first value, increase the set air volume, and
based on the temperature difference being greater than or equal to a predetermined second value, decrease the set air volume.

9. The mask of claim 1, further comprising:
a filter arranged on an air flow channel through the fan, the inside of the mask, and the valve,
wherein the filter comprises:
a first filter arranged upstream from a location of the fan; and
a second filter arranged downstream than the location of the fan and upstream from a location of the valve.

10. A controlling method for a mask, the method comprising:
detecting pressure including a maximum pressure value and a minimum pressure value inside the mask worn by a user;
identifying a time for a single breath based on the detected point of the maximum pressure value and the detected point of the minimum pressure value, and identifying a number of breaths based on the identified time for the single breath and a first predetermined time; and
providing an air volume set at a level corresponding to the identified number of breaths among a plurality of levels that are divided based on a predetermined number of breaths to an inside of the mask.

11. The controlling method for the mask of claim 10, wherein the identifying the number of breaths comprises:
based on identifying an abnormality in the detected pressure, identifying the number of breaths based on the number of opening and closing of a valve.

12. The controlling method for the mask of claim 11, wherein the identifying the number of breaths comprises:
based on the detected maximum pressure value or the detected minimum pressure value exceeding a predetermined normal breathing range, identifying that there is an abnormality in the detected pressure.

13. The controlling method for the mask of claim 12, further comprising:
based on the identified number of breaths lasting for a second predetermined time is below a predetermined minimum value, turning off power to a fan.

14. The controlling method for the mask of claim 10, further comprising:
based on receiving information of changing the predetermined number of breaths dividing the plurality of levels from a terminal device, changing the predetermined number of breaths based on the received information.

15. The controlling method for the mask of claim 10, further comprising:
receiving an external temperature of a region wherein the user is located from a terminal device;
detecting an internal temperature of the mask; and
changing a set air volume based on a temperature difference between the received external temperature and the detected internal temperature.

16. The controlling method for the mask of claim 10, further comprising:
detecting an external temperature of the mask and an internal temperature of the mask, and
changing a set air volume based on a temperature difference between the detected external temperature and the detected internal temperature.

17. The controlling method for the mask of claim 16, further comprising:
based on the temperature difference being smaller than or equal to a predetermined first value, increasing the set air volume, and
based on the temperature difference being greater than or equal to a predetermined second value, decreasing the set air volume.

18. The controlling method for the mask of claim 10, further comprising:
arranging a filter on an air flow channel through a fan, the inside of the mask, and a valve,
wherein the filter comprises:
a first filter arranged upstream from a location of the fan; and
a second filter arranged downstream from the location of the fan and upstream from a location of the valve.

19. A controlling method for a mask, the method comprising:
acquiring an internal temperature and an external temperature of the mask worn by a user; and
controlling an air volume provided to an inside of the mask based on a temperature difference between the acquired internal temperature and the acquired external temperature.

20. The controlling method for the mask of claim 19, further comprising:
based on the temperature difference being smaller than or equal to a predetermined first value, increasing the air volume, and
based on the temperature difference being greater than or equal to a predetermined second value, decreasing the air volume.
